(19) United States
(12) Patent Application Publication
(10) Pub. No.: US 2020/0046620 A1
(43) Pub. Date: Feb. 13, 2020

(54) Solid Compositions for Teeth Whitening and Anti-Microbial Effects

(71) Applicant: Colgate-Palmolive Company, New York, NY (US)

(72) Inventors: Chun HUANG, Somerset, NJ (US); Paloma PIMENTA, Staten Island, NY (US); Luciana RINAUDI MARRON, Somerset, NJ (US); Indrani BANERJEE, Harrison, NJ (US); Rong DONG, Highland Park, NJ (US)

(73) Assignee: Colgate-Palmolive Company, New York, NY (US)

(21) Appl. No.: 16/536,474

(22) Filed: Aug. 9, 2019

Related U.S. Application Data
(60) Provisional application No. 62/717,022, filed on Aug. 10, 2018.

(51) Int. Cl. A61K 8/49; A61K 8/02; A61K 8/19; A61K 8/24; A61Q 11/00

(52) U.S. Cl. CPC A61K 8/4966; A61K 8/0216; A61K 8/19; A61K 2800/222; A61Q 11/00; A61K 2800/88; A61K 2800/92; A61K 8/24

(57) ABSTRACT

Disclosed herein is a solid composition for oral care comprising at least one whitening agent chosen from chloride isocyanurate and metal salts of hypochlorite; at least one carbonate base; and optionally at least one effervescent acid. Further disclosed herein is a kit comprising the solid composition and an aqueous liquid composition, as well as methods of whitening teeth and/or producing antibacterial effects in the oral cavity comprising mixing the solid composition with an aqueous liquid composition until the solid composition disintegrates to create a mouthwash comprising hypochlorite; and contacting teeth with the mouthwash comprising hypochlorite.

### BACKGROUND

Many consumers have a desire for oral care products that have anti-microbial effects, such as plaque-reducing effects. Dental plaque is a soft deposit that forms on teeth and comprises an accumulation of bacteria and bacterial by-products. Besides being unsightly, plaque is implicated in the occurrence of gingivitis and other forms of periodontal disease. Cationic anti-bacterial agents (for example, arginine-based esters, Sn (II) compounds, and cetylpyridinium chloride) have been proposed for use in oral care compositions, and in particular, to counter plaque formation and oral infections associated with plaque formation. However, these agents are often incompatible with other ingredients found in mouthwash formulations, reducing their anti-microbial activity in such formulations.

Many consumers also desire whiter teeth, which can be achieved through the use of tooth-whitening products. The whitening effect can be produced chemically by altering or removing the stain and/or changing the visual perception of the color of the teeth.

Peroxide compounds (for example, hydrogen peroxide (H₂O₂)) can provide whitening benefit in mouthwash formulations. However, these formulations potentially lose their whitening efficacy over time as peroxide compounds in aqueous solutions are relatively unstable. Furthermore, peroxide compounds are incompatible with many active oral care agents, including anti-microbial compounds. For example, SnF₂ (Sn II) has anti-bacterial activity in the absence of peroxide compounds; however, SnF₂ is unstable in the presence of peroxides and undergoes oxidation to Sn (IV). Other cationic antibacterial agents such as cetylpyridinium chloride may stain teeth and therefore reduce the whitening efficacy of peroxide compounds.

Because of the shelf stability requirements of hydrogen peroxide in formulations, most teeth whitening products are formulated at a low pH between about 4.5 to about 5.5. This may also lead to poor whitening efficacy because shelf-stable hydrogen peroxide also has a lower oxidation potential. Another issue for products comprising hydrogen peroxide is the governmental regulation of hydrogen peroxide in oral care products. For example, in Europe, hydrogen peroxide concentration has to be kept less than 0.1% for over-the-counter products.

Although other bleaching and/or antimicrobial agents are known, they may not be stable in aqueous solutions or at lower pHs, and therefore are not used in oral care compositions such as mouthwashes. For example, hypochlorite is known to be both an efficient bleaching and antimicrobial agent. Hence, a composition comprising hypochlorite could be used for oral care to provide a teeth whitening benefit as well as an anti-microbial benefit. However, hypochlorite is not stable in aqueous environments at a pH of less than about 13; therefore, it cannot be directly added to many oral care compositions, such as mouthwashes. In order to avoid the stability issue of using hypochlorite-based chemicals directly in a liquid composition, precursor molecules, such as sodium dichloroisocyanurate (NaDCC) have been used to generate hypochlorite during usage by triggering the conversion of NaDCC to hypochlorite when it comes into contact with water. Such materials have been used in drinking water treatment, which gives the opportunity to use them as ingredients in oral care products without safety concern. Per World Health Organization regulations, the daily intake limit for adults for NaDCC, for example, is 2.2 mg/day/kg weight.

Therefore, it would be desirable to provide a stable, safe, and effective oral care composition that has both whitening activity and anti-microbial activity.

### BRIEF SUMMARY

Disclosed herein is a solid composition for oral care, such as for providing teeth whitening and/or antibacterial effect, comprising at least one whitening agent chosen from chloride isocyanurate and metal salts of hypochlorite, at least one carbonate base, and optionally at least one effervescent acid. In certain embodiments, the at least one carbonate base is present in the solid composition in an amount ranging from about 45% to about 95%, such as about 65% to about 80%, by weight relative to the total weight of the solid composition, and in certain embodiments, the at least one effervescent acid is present in the solid composition in an amount ranging from about 0.01% to about 20%, such as about 1% to about 5%, by weight relative to the total weight of the composition. In certain embodiments, provided herein is a solid composition for teeth whitening comprising at least one whitening agent chosen from chloride isocyanurate and metal salts of hypochlorite and at least one carbonate base present in the solid composition in an amount ranging from about 45% to about 95%, wherein the solid composition is free of an effervescent acid.

According to various embodiments of the disclosure, the at least one whitening agent is chloride isocyanurate, such as sodium dichloroisocyanurate, and in certain embodiments, the chloride isocyanurate is present in the solid composition in an amount ranging from about 0.5% to about 50%, such as about 10% to about 30%, or about 20%, by weight relative to the total weight of the solid composition. According to various embodiments of the disclosure, the at least one carbonate base is sodium bicarbonate, and in certain embodiments, the at least one effervescent acid is sodium acid pyrophosphate.

In certain embodiments of the disclosure, the solid composition is a tablet, and in certain embodiments, the solid composition is anhydrous. Additionally, in certain exemplary embodiments, the at least one solid composition is free of microcrystalline cellulose, free of magnesium stearate, and/or free of polymers, such as water-soluble polymers. In certain other embodiments, the solid composition comprises at least one water-soluble polymer, such as at least one water-soluble polymer chosen from polyvinylpyrrolidone and polyvinylpyrrolidone vinyl acetate.

According to certain embodiments disclosed herein, about 50 mg of the solid composition is capable of disintegrating in about 20 mL of an aqueous liquid composition, such as a mouthwash, in less than about 180 seconds, such as less than about 90 seconds, or about 15 seconds or less.

Also disclosed herein is a kit comprising (1) a solid composition for oral care comprising at least one whitening agent chosen from chloride isocyanurate and metal salts of hypochlorite, at least one carbonate base present in the solid composition in an amount ranging from about 45% to about 95% by weight relative to the total weight of the solid composition, and at least one effervescent acid present in an amount ranging from about 0.01% to about 20% by weight relative to the total weight of the solid composition; and (2) a container comprising an aqueous mouthwash composition. In certain embodiments, the kit further comprises a cup suitable for holding a specific amount, such as about 20 mL or about 15 mL, of the aqueous mouthwash composition and a unit dose of the solid composition, and in certain embodiments, the at least one whitening agent in the solid composition in the kit is sodium dichloroisocyanurate. In certain embodiments there is provided a kit comprising (1) a solid composition for oral care comprising at least one whitening agent chosen from chloride isocyanurate and metal salts of hypochlorite and at least one carbonate base present in the solid composition in an amount ranging from about 45% to about 95% by weight relative to the total weight of the solid composition; and (2) a container comprising an aqueous mouthwash composition comprising at least one effervescent acid. In certain embodiments of the kit disclosed herein, when the solid composition is combined with a unit dosage of the aqueous mouthwash composition, the at least one effervescent acid may be present in the unit dosage of the aqueous mouthwash composition in an amount ranging from about 0.01% to about 20% by weight relative to the weight of the solid composition.

Further disclosed herein is a method of whitening teeth comprising (1) mixing a solid composition for oral care comprising at least one whitening agent chosen from chloride isocyanurate and metal salts of hypochlorite, at least one carbonate base present in the solid composition in an amount ranging from about 45% to about 95%, and at least one effervescent acid present in an amount ranging from about 0.01% to about 20% with an aqueous liquid composition until the solid composition disintegrates to create a mouthwash comprising hypochlorite; (2) contacting teeth with the mouthwash comprising the hypochlorite; and (3) optionally expectorating the mouthwash comprising hypochlorite. In other embodiments of the disclosure, there is provided a method of whitening teeth comprising (1) mixing a solid composition for oral care comprising at least one whitening agent chosen from chloride isocyanurate and metal salts of hypochlorite and at least one carbonate base present in the solid composition in an amount ranging from about 45% to about 95% with an aqueous liquid composition comprising at least one effervescent acid until the solid composition disintegrates to create a mouthwash comprising hypochlorite; (2) contacting teeth with the mouthwash comprising the hypochlorite; and (3) optionally expectorating the mouthwash comprising hypochlorite. In certain embodiments of the method disclosed herein, about 50 mg of the solid composition disintegrates in about 20 mL of the aqueous liquid composition in less than about 180 seconds, such as less than about 90 seconds, less than about 60 second, about 15 second or less, or about 10 seconds or less.

Further areas of applicability of the present disclosure will become apparent from the detailed description provided hereinafter. It should be understood that the detailed description and specific examples, while indicating certain preferred embodiments of the disclosure, are intended for purposes of illustration only and are not intended to limit the scope of the disclosure.

### DETAILED DESCRIPTION

The following description of the preferred embodiments is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

As used herein, the term “one or more of” with respect to a listing of items such as, for example, A and B, means A alone, B alone, or A and B. The term “at least one of” is used to mean one or more of the listed items can be selected.

Unless otherwise specified, all percentages and amounts expressed herein and elsewhere in the specification should be understood to refer to percentages by weight. The amounts given are based on the active weight of the material.

Disclosed herein is a solid composition, such as a tablet or powder, comprising at least one whitening agent chosen from chloride isocyanurate and metal salts of hypochlorite, at least one carbonate base, and optionally at least one effervescent acid. The solid composition may be quick-disintegrating, such that it can be added to a liquid aqueous composition, such as water, saliva, or a mouthwash, to be used by a consumer to provide teeth whitening and/or antibacterial benefits.

The solid compositions disclosed herein comprise at least one at least one whitening agent chosen from chloride isocyanurate and metal salts of hypochlorite. Non-limiting examples of suitable chlorinated isocyanurates include anhydrous sodium dichloroisocyanurate (NaDCC), sodium dichloroisocyanurate dihydrate (NaDCC₂H₂O), potassium dichloroisocyanurate (KDCC), trichloroisocyanuric acid (TCCA), and calcium dichloroisocyanurate, as well as other dichloroisocyanurate salts, hydrates, and combinations thereof. In certain embodiments, the chlorinated isocyanurate comprises NaDCC. NaDCC is generally of the chemical formula C₃Cl₂N₃NaO₃, CAS Number 2893-78-9, and may also be referred to as sodium dichloro-s-triazine-trione, sodium 3,5-dichloro-2,4,6-trioxo-1,3,5-triazinan-1-ide, sodium troclosene, sodic troclosene, troclosenum natricum, dichloroisocyanuric acid, or sodium salt of dichloroisocyanuric acid. NaDCC is shown below as Formula I:

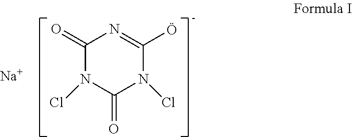

In certain embodiments, the at least one whitening agent is chosen from metal salts of hypochlorite. Non-limiting examples of suitable metal salts of hypochlorite include lithium hypochlorite, potassium hypochlorite, sodium hypochlorite, magnesium hypochlorite, calcium hypochlorite, barium hypochlorite, and mixtures thereof.

The at least one whitening agent chosen from chloride isocyanurate and metal salts of hypochlorite disclosed herein may be present in the solid composition in any amount effective for teeth whitening and/or antibacterial benefits. For example, the amount of at least one whitening agent chosen from chloride isocyanurate and metal salts of hypochlorite in the solid compositions disclosed herein may be effective to result in improved tooth whitening when used in a mouthwash as compared to a control mouthwash without the whitening agent. Additionally, the amount of at least one whitening agent chosen from chloride isocyanurate and metal salts of hypochlorite in the solid compositions disclosed herein may be effective to result in antibacterial activity in the oral cavity of a user. The amount of the at least one whitening agent chosen from chloride isocyanurate and metal salts of hypochlorite may, in certain embodiments, range from about 0.5% to about 50%, such as about 5% to about 40%, about 7.5% to about 37%, about 10% to about 25%, or about 20%, by weight relative to the total weight of the solid composition. In certain embodiments, the chlorinated isocyanurate is NaDCC present in an amount ranging from about 1% to about 25%, such as about 20%, by weight relative to the total weight of the solid composition. In certain embodiments, the amount of NaDCC present in the solid composition is such that a unit dose of the solid composition does not exceed 2.2 mg of NaDCC per day per kg weight of the user.

Chlorinated isocyanurates and such as NaDCC and metal salts of hypochlorite such as sodium hypochlorite may not be stable in the presence of water. Rather, when contacted with water, NaDCC converts to hypochlorite. The chemistry below illustrates the conversion of NaDCC to hypochlorite:

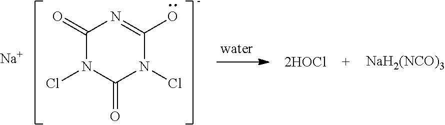

As discussed above, hypochlorite, like other forms of chloride, is a known bleaching agent and disinfectant. Disclosed herein is a method of using at least one of chloride isocyanurate and metal salts of hypochlorite in an oral composition for teeth whitening and/or antibacterial effect comprising generating hypochlorite in situ by mixing the solid compositions disclosed herein into an aqueous liquid composition, such as water, saliva, or a mouthwash. The chloride isocyanurate or metal salt of hypochlorite in the solid composition releases hypochlorite following exposure to water. By using the approach of a solid composition (such as a tablet) plus an aqueous liquid composition, such as a mouthwash, whitening and/or antibacterial efficacy may be improved while the mouthwash formula shelf stability is maintained, since the active ingredient, hypochlorite, is generated at the point of use, when the solid composition is mixed with the mouthwash.

The solid compositions disclosed herein further comprise at least one carbonate base. Examples of suitable carbonate bases include sodium bicarbonate, sodium carbonate, sodium sesquicarbonate, potassium carbonate, potassium bicarbonate, calcium carbonate, magnesium carbonate, magnesium oxide, sodium glycine carbonate, L-lysine carbonate, arginine carbonate, zinc carbonate, zinc oxide and mixtures thereof. The at least one carbonate base may be present in the compositions disclosed herein in any effective amount. In certain embodiments, the at least one carbonate base is present in the solid composition in an amount ranging from about 45% to about 95%, such as about 65% to about 80% or about 72% to about 78%, by weight relative to the total weight of the solid composition.

The solid compositions disclosed herein may further comprise at least one effervescent acid. The solid composition of the invention is optionally an effervescent composition. The term “effervescent composition” as used herein indicates a composition that produces gas bubbles, such as carbon dioxide bubbles, when contacted with water. When the solid composition disclosed herein is an effervescent composition, it may comprise an effervescent agent. The effervescent agent may be an effervescent couple that includes an acid and a base. The effervescent couple is activated when contacted with water, such as when the solid composition is placed in a container of an aqueous liquid composition, such as water or a mouthwash. The water liberates the effervescent acid and the base, enabling the acid and base to react with each other to produce carbon dioxide gas, which imparts carbonation to the aqueous liquid composition. Examples of effervescent acids that may be mentioned include acids and their acid anhydrides and salts, such as citric acid, ascorbic acid, malic acid, adipic acid, tartaric acid, fumaric, succinic acid, sodium acid pyrophosphate, lactic acid, hexamic acid, citraconic anhydride, glucono-D-lactone, succinic anhydride, potassium bitartrate, acid citrate salts, sodium dihydrogen phosphate, disodium dihydrogen phosphate, sodium acid sulfite, and combinations thereof. The at least one effervescent acid may be present in the solid composition in an amount ranging from about 0% to about 50%, such as from about 0.01% to about 20%, about 0.1% to about 10%, about 1.5% to about 8%, or about 2% to about 7.5%, by weight relative to the total weight of the solid composition. In certain embodiments, the at least one effervescent acid is sodium pyrophosphate. In certain embodiments of the disclosure, the at least one carbonate and the at least one effervescent acid are present in a ratio of greater than about 1:1, such as greater than about 2:1, greater than about 10:1, greater than about 15:1, or greater than about 30:1.

In certain other embodiments of the disclosure, the solid compositions may be free of an effervescent acid, such as free of adipic acid. When the solid composition is free of an effervescent acid, the solid composition may, in certain embodiments, be combined with an aqueous composition comprising at least one effervescent acid, such as an aqueous mouthwash composition comprising at least one effervescent acid. When the solid composition that is free of an effervescent acid is combined with the aqueous composition comprising at least one effervescent acid, the at least one effervescent acid and the at least one carbonate base may combine to allow the solid composition to disintegrate in the aqueous composition.

The solid compositions disclosed herein may be anhydrous, meaning the compositions are free or substantially free of water. As used herein, the term “free or substantially free of” a substance means the composition comprises no effective amount of that substance, such as no effective amount of water. In various embodiments of the solid composition, the amount of water is in an amount of less than less than 1% by weight, such as less than 0.5% by weight, about 0.001% to about 4% by weight, about 0.0001% to about 0.5% by weight, or about 0% to about 0.1% by weight, by weight based on the total weight of the composition.

The solid compositions disclosed herein may optionally contain at least one disintegrating agent, for example, when the composition is a tablet, such as an effervescent tablet. Disintegrating agents may include natural starches, such as maize starch, potato starch etc., directly compressible starches such as starch 1500, modified starches such as carboxymethyl starches and sodium starch glycolate which are available as Primojel®, Explotab®, and Explosol® and starch derivatives such as amylose. Other examples may include cross-linked polyvinylpyrrolidones, such as crospovidones available, for example, as Polyplasdone® XL and Kollidon® XL; modified celluloses such as cross-linked sodium carboxymethylcelluloses available as, for example, Ac-di-sol®, Primellose®, Pharmacel® XL^{T}, Explocel®, and Nymcel® ZSX; alginic acid and sodium alginate; microcrystalline cellulose, such as Avicel®, Pharmacel®, Emcocel®, Vivapur®; and methacrylic acid-divinylbenzene copolymer salts available, for example, as Amberlite® IRP-88. Other examples of disintegrating agents may include light silicic anhydride, calcium silicate, magnesium metasilicate aluminate, and carboxymethyl cellulose. The amount of the at least one disintegrating agent may range from about 0% to about 20%, such as about 1% to about 5%, or about 1% to about 3%, by weight relative to the total weight of the composition. In certain embodiments, the at least one disintegrating agent is microcrystalline cellulose. In certain embodiments, the solid composition is free of a disintegrating agent, an in certain embodiments, the solid composition is free of microcrystalline cellulose.

The solid compositions disclosed herein may optionally comprise at least one lubricant. Various lubricants are suitable for use in the composition including water-dispersible, water-soluble, and water-insoluble lubricants and combinations thereof. Examples of useful water soluble lubricants may include sodium benzoate, polyethylene glycol, L-leucine, adipic acid, and combinations thereof. The composition can also include water-insoluble lubricants including, for example, stearates such as magnesium stearate, calcium stearate, and zinc stearate; oils such as mineral oil, hydrogenated and partially hydrogenated vegetable oils, and cotton seed oil; animal fats; polyoxyethylene monostearate; talc; and combinations thereof. When the solid composition is in the form of a tablet, the composition may include a sufficient amount of lubricant to enable the composition to be formed into tablets and released from a high speed tableting press in the form of a tablet. In certain embodiments, the amount of lubricant in the composition may range from about 0% to about 15%, such as from about 0.5% to about 10%, from about 1% by weight to about 5% by weight, or from about 1.5% to about 2.5%, by weight relative to the total weight of the solid composition. In one embodiment, the solid composition is free from a lubricant, and in one embodiment, the solid composition is free from magnesium stearate. In certain other embodiments, the solid composition comprises at least about 0.5%, at least about 1%, or at least about 1.5% magnesium stearate.

According to various embodiments of the disclosure, the solid compositions disclosed herein may further comprise at least one water-soluble polymer. Exemplary water-soluble polymers that may be mentioned include cellulose ethers, methacrylates, polyvinylpyrollidone, and mixtures thereof. In certain embodiments, the water-soluble polymer may be a cellulose ether, including those selected from the group consisting of hydroxyalkyl cellulose polymers such as hydroxypropyl methyl cellulose (HPMC), hydroxypropyl cellulose, hydroxyethyl cellulose, methyl cellulose, carboxymethyl cellulose, and mixtures thereof. Other polymers among those useful herein include polyvinylpyrrolidone (PVP), cross-linked polyvinyl pyrrolidone, polyvinylpyrrolidone-vinyl acetate (PVP-VA) copolymer, polyvinylalcohol, polyacrylic acid, poly acrylate polymer, cross-linked polyacrylate polymer, cross-linked polyacrylic acid (such as Carbopol®), polyethylene oxide, polyethylene glycol, poly vinyl alkyl ether-maleic acid copolymer (such as Gantrez®) and carboxy vinyl polymer; natural gums such as sodium alginate, carrageenan, xantham gum, gum acacia, arabic gum, guar gum, pullulan, agar, chitin, chitosan, pectin, karaya gum, zein, hordein, gliadin, locust bean gum, tragacantha and other polysaccharides; starches such as maltodextrin, amylose, high amylose starch, corn starch, potato starch, rice starch, tapioca starch, pea starch, sweet potato starch, barley starch, wheat starch, waxy corn starch, modified starch (such as hydroxypropylated high amylose starch), dextrin, levan, elsinan and gluten; and proteins such as collagen, whey protein isolate, casein, milk protein, soy protein and gelatin. In certain embodiments, the solid composition disclosed herein may be free of water-soluble polymers, such as free of PVP and PVP-VA.

The solid compositions disclosed herein may optionally comprise at least one whitening agent in addition to the chloride isocyanurate and metal salts of hypochlorite. In various embodiments, the compositions disclosed herein may optionally comprise a peroxide whitening agent, comprising a peroxide compound. A peroxide compound is an oxidizing compound comprising a bivalent oxygen-oxygen group. Peroxide compounds include peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, monoperoxyphthalate, peroxymonosulfate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound comprises hydrogen peroxide, urea peroxide, sodium percarbonate and mixtures thereof. In some embodiments, the peroxide compound comprises hydrogen peroxide. In some embodiments, the peroxide compound consists essentially of hydrogen peroxide. In some embodiments a non-peroxide whitening agent may be provided. Non-peroxide whitening agents may include, for example, colorants such as titanium dioxide and hydroxyapatite. One or more additional whitening agents are optionally present in a tooth-whitening effective total amount. In some embodiments, the solid compositions disclosed herein additionally comprise at least one activator, such as tetraacetylethylenediamine.

The solid compositions disclosed herein may optionally comprise any other ingredients known in the art, including for example, fillers; surfactants; flavor agents; preservatives, such as sodium benzoate and potassium sorbate; coloring agents; and sweeteners.

Examples of fillers include crystalline cellulose, ethylcellulose, dextrin, various kinds of cyclodextrin (α-cyclodextrin, ß-cyclodextrin and γ-cyclodextrin), pullulan, and sodium sulfate, as well as derivatives thereof.

Examples of surfactants that can be used include sodium lauryl sulfate, sorbitan fatty acid ester, polyoxyethylene (20) sorbitan monooleate (Polysorbate 80 or Tween® 80), polyethylene glycol fatty acid ester, polyoxyethylene sorbitan fatty acid ester, polyoxyethylene alkyl ether, polyoxyethylene polyoxypropylene alkyl ether, polyoxyethylene polyoxypropylene block copolymer, polyoxyethylene alkyl phenyl ether, polyoxyethylene castor oil, polyoxyethylene hydrogenated castor oil, polyoxyethylene sorbitol fatty acid ester and polyoxyethylene glycerol fatty acid ester. In embodiments wherein at least one surfactant is included in the solid compositions disclosed herein, the surfactant(s) may be present in an amount ranging from about 0.5% to about 3%, such as about 0.75% to about 2%, or about 1% to about 1.5%, by weight relative to the total weight of the solid composition.

Exemplary flavor agents that may be mentioned include natural and synthetic flavoring sources including volatile oils, synthetic flavor oils, flavoring aromatics, oils, liquids, oleoresins and extracts derived from plants, leaves, flowers, fruits, stems and combinations thereof. Flavor agents may include, for example, citric oils, such as lemon, orange, grape, lime and grapefruit; fruit essences such as apple, pear, peach, grape, strawberry, raspberry, cherry, plum, pineapple, apricot; and other fruit flavors. Other useful flavor agents include, for example, aldehydes and esters (such as benzaldehyde (cherry, almond)), citral, i.e., α-citral (lemon, lime), neral, i.e., ß-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin), and mixtures thereof.

Exemplary coloring agents may include food, drug and cosmetic (FD&C) colors including, for example, dyes, lakes, pigments, and certain natural and derived colorants. Lakes include dyes absorbed on aluminum hydroxide and other suitable carriers.

Exemplary sweetening agents may include stevia; sugars such as sucrose, glucose, invert sugar, fructose, ribose, tagalose, sucralose, maltitol, erythritol, xylitol, and mixtures thereof; saccharin and its various salts (e.g., sodium and calcium salt of saccharin); cyclamic acid and its various salts; dipeptide sweeteners (e.g., aspartame); acesulfame potassium; dihydrochalcone; glycyrrhizin; and sugar alcohols including, for example, sorbitol, sorbitol syrup, mannitol and xylitol, and combinations thereof.

The solid composition of the invention can be in a variety of forms including, e.g., powder (e.g., a free flowing granulation), tablet, caplet, granule, pellet, wafer, film and bead.

The solid compositions disclosed herein, such as tablets, can be formed to have any desired weight and dimension. For example, tablets may weight from about 0.05 grams to about 5 grams, such as from about 20 mg to about 1000 mg, from about 40 mg to about 500 mg, or from about 50 mg to about 100 mg. Tablets may also, for example, be formed with a diameter of at least about 1 mm, such as from about 1 mm to about 100 mm or from about 5 mm to about 50 mm, and a thickness of at least about 0.5 mm, such as from about 1 mm to about 20 mm or from 5 mm to about 15 mm. In some embodiments, the surface area of the solid compositions, such as tablets or beads, may be, for example, from about 0.55 cm² to about 9.5 cm², such as from about 0.9 cm² to about 5 cm².

Because the solid compositions disclosed herein, such as tablets, are not stable in the presence of water, the composition may be packaged in a moisture-free environment. In certain embodiments, the solid composition may be individually packaged and sealed in a unit dose. As used herein, the term “unit dose” indicates an amount of the solid composition intended for one use. The solid composition may be stored in an air-tight, moisture-proof package, including, for example, sealed metal foil pouches, blister packs, and tubes, such as desiccant-capped tubes.

According to certain embodiments, am amount, such at least about 20 mg, at least about 30 mg, at least about 40 mg, at least about 50 mg, at least about 75 mg or at least about 100 mg, of the solid composition disclosed herein may completely disintegrate in 20 mL of an aqueous liquid composition, such as water or a mouthwash, at room temperature in about 500 seconds or less, such as less than about 400 seconds, less than about 360 seconds, less than about 180 seconds, less than about 120 seconds, less than about 60 seconds, less than about 45 seconds, less than about 30 seconds, about 15 seconds or less, or about 10 second or less. In certain embodiments, the solid composition disclosed herein may completely disintegrate in saliva present in the oral cavity at body temperature in about in about 500 seconds or less, such as less than about 400 seconds, less than about 360 seconds, less than about 180 seconds, less than about 120 seconds, less than about 60 seconds, less than about 45 seconds, less than about 30 seconds, about 15 seconds or less, or about 10 second or less. As used herein, the phrase “completely disintegrates” means that the solid composition completely breaks apart into particulate matter, such as a fine powder. In certain embodiments, agitation may be used to disintegrate the solid composition. As used herein, “agitation” means shaking or swirling or stirring of the mixture by hand or with the use of an implement, by the consumer in a suitable container such as a cup.

The solid compositions disclosed herein can be made via techniques known in the art. In certain embodiments, the ingredients can be kneaded with an organic solvent, filled in a mold, and subjected to a compression-molding. The organic solvent can be an alcohol such as methanol, ethanol, propanol, isopropanol, and the like. The kneading and granulating operations carried out by adding such auxiliary agents for making the preparation and by adding such a solvent may be conducted using any conventionally-used apparatus. For example, a fluidized bed granulator, a tumbling granulator, an extrusion granulator, or a spray-drying drier may be used. The solid compositions may also be prepared via freeze drying. Powders can be prepared by compounding the ingredients and optionally calcium carbonate, and, if necessary, further orally acceptable additives, and mixing in a conventional manner. Granules can be prepared by any one of known methods for preparing granules such as dry granulation, layering granulation, impregnated-granulation, etc.

Tablets can be manufactured by either subjecting a mixture prepared in the same manner as above to the compression molding as it is, or subjecting said mixture to the granulation as mentioned above, and then to the compression molding after adding, for example, any desired disintegrants, lubricants, etc. If a carbonate is compounded, it may be added at the same time when as any optional disintegrants and/or lubricants are added. The compression molding can be conducted using a conventional tableting machine, such as rotary tableting machines, single punch tableting machines, dual tableting machines, and the like, with a compressing pressure of generally about 50 to 4,000 kg/cm².

Further disclosed herein are kits comprising a solid composition as disclosed herein. In certain embodiments, a kit comprises (1) a solid composition comprising at least one whitening agent chosen from chloride isocyanurate and metal salts of hypochlorite, at least one carbonate base, and at least one effervescent acid; and (2) an aqueous liquid composition, such as a mouthwash. In certain other embodiments, disclosed herein are kits comprising (1) a solid composition comprising at least one whitening agent chosen from chloride isocyanurate and metal salts of hypochlorite and at least one carbonate base; and (2) an aqueous liquid composition, such as a mouthwash, comprising at least one effervescent acid. According to various embodiments, the kits disclosed herein may comprise multiple unit doses of the solid composition, individually packaged in moisture-free package or packaged together with multiple unit doses in a single air-tight resealable container. In certain embodiments, the kit further comprises a cup capable of holding a single unit dose of the aqueous liquid composition, such as capable of holding about 15 mL or about 20 mL of the aqueous liquid composition, together with a single unit dose of the solid composition.

The terms “mouthwash” or “mouthrinse” generally denote liquid formulations that are used to rinse the surfaces of the oral cavity and provide the user with a sensation of oral cleanliness and refreshment. The mouthwash is an oral composition that is not intentionally swallowed for purposes of systemic administration of therapeutic agents, but is applied to the oral cavity, used to treat the oral cavity, and then optionally expectorated.

An aqueous mouthwash composition may comprise a liquid carrier such as water; humectants, such as glycerin, sorbitol, and propylene glycol; surfactants, such as a Pluronics® and sodium lauryl sulfate; sweetening agents, such as sodium saccharin and xylitol; flavoring agents; coloring agents, preservative agents, such as potassium sorbate and sodium benzoate; buffering agents such as sodium phosphates; anti-cavity agents, such as sodium fluoride; and anti-bacterial agents such as cetylpyridinium chloride. In certain embodiments the aqueous mouthwash compositions disclosed herein may have a pH ranging from about 4 to about 7, such as a pH of about 4.5. In certain embodiments, the aqueous mouthwash composition may comprise at least one effervescent acid as described above.

Further disclosed herein is a method for whitening teeth comprising mixing a unit dose of the solid composition comprising at least one whitening agent chosen from chloride isocyanurate and metal salts of hypochlorite into an aqueous liquid composition, such as water or a mouthwash, until the solid composition disintegrates in the liquid to form a mouthwash solution comprising hypochlorite, followed by contacting teeth with the mouthwash solution comprising hypochlorite. In certain embodiments, the method comprises contacting the teeth by the user putting the mouthwash solution comprising hypochlorite into the oral cavity, and swirling or swishing the mouthwash around the oral cavity for a period of time before optionally expectorating the mouthwash. In certain embodiments, the mouthwash may be in the oral cavity contacting the teeth for a period of at least about 10 seconds, such as at least about 20 seconds, at least about 30 seconds, at least about 45 seconds, at least about 1 minute, or at least about 2 minutes. In order to achieve maximum whitening efficacy, the methods disclosed herein may further comprise repeating the method for whitening teeth multiple times. In certain embodiments, the method may be repeated at least once a day, such as twice a day, for a period of time such as 5 days, 10 days, 2 weeks, or 1 month. In certain embodiments, the method for whitening teeth may be repeated as necessary.

In the methods for whitening teeth disclosed herein, the whitening efficacy of a composition may be measured, for example, using the CIE L*a*b* (CIELAB) scale developed by the International Commission on Illumination (CIE). CIELAB is an opponent color system based on the fact that retinal color stimuli are translated into distinctions between light and dark, red and green, and blue and yellow. CIELAB indicates these values with three axes: L*, a*, and b*. The L value indicates the lightness of a color, where L=0 is black and L=100 is white. ΔL=L_{after treatment}−Lᵢₙᵢₜᵢₐₗ. Thus, a larger positive ΔL value indicates whiter teeth. The a value ranges between +a=magenta and −a=green. The b value ranges between +b=yellow and −b=blue. The W value incorporates the L, a and b values to describe how close the measured color is to true white, where W*=(a²+b²+(L*−100)²)^{1/2}. A larger negative ΔW value corresponds to greater whitening.

Further disclosed herein is a method for producing an antibacterial effect comprising mixing a unit dose of the solid composition disclosed herein comprising at least one whitening agent chosen from chloride isocyanurate and metal salts of hypochlorite into an aqueous liquid composition, such as water or a mouthwash, until the solid composition disintegrates in the liquid to form an antibacterial solution comprising hypochlorite, followed by contacting the oral cavity with the antibacterial solution comprising hypochlorite. In certain embodiments, the method comprises contacting the teeth by the user putting the antibacterial solution into the oral cavity, and swirling or swishing the antibacterial solution around the oral cavity for a period of time before optionally expectorating the antibacterial solution. In certain embodiments, the antibacterial solution may be in the oral cavity for a period of at least about 10 seconds, such as at least about 20 seconds, at least about 30 seconds, at least about 45 seconds, at least about 1 minute, or at least about 2 minutes. In order to achieve maximum antibacterial efficacy, the methods disclosed herein may further comprise repeating the method for producing an antibacterial effect multiple times. In certain embodiments, the method may be repeated at least once a day, such as twice a day; in certain embodiments, the method may be repeated for a period of time such as 5 days, 10 days, 2 weeks, 1 month, daily, weekly, monthly, or as needed. Antibacterial effects may include, by way of non-limiting examples, at least one of reducing or inhibiting the formation of dental caries, gingivitis, malodor of the breath, microbial biofilm formation in the oral cavity, and accumulation of plaque.

### EXAMPLES

### Example 1—Teeth Whitening Evaluation

A mouthwash formula having a final pH of 4.33 was prepared as shown below in Table 1, and four anhydrous tablets (Tablets A, B, C, and D) comprising NaDCC were prepared as shown below in Table 2. All of the tablets contained 40 mg of sodium bicarbonate, and Tablets A, B, C, and D contained 20% NaDCC, 11.1% NaDCC, 4.8% NaDCC, and 0% NaDCC, respectively.

TABLE 1
Mouthwash formulation

| Ingredients | Weight (%) |
|---|---|
| Water | 83.18 |
| Sorbitol | 5 |
| Propylene glycol | 7.05 |
| Poloxomer 407 | 0.5 |
| Sodium saccharin | 0.05 |
| Methyl levo | 0.05 |
| Methyl salicylate | 0.17 |
| Sodium acid | 4 |
| pyrophosphate | |
| (SAPP) | |

TABLE 2
Tablet formulation

| | Diameter | Height | | Amount |
|---|---|---|---|---|
| Tablet | (mm) | (mm) | Ingredients | (mg) |
| A | 5.0 | 1.12 | NaDCC | 10 mg |
| | | | NaHCO₃ | 40 mg |
| B | 5.0 | 0.99 | NaDCC | 5 mg |
| | | | NaHCO₃ | 40 mg |
| C | 5.0 | 0.94 | NaDCC | 2 mg |
| | | | NaHCO₃ | 40 mg |
| D | 5.0 | 0.87 | NaHCO₃ | 40 mg |

The tablets were made using a homemade press and die set with a diameter of 5 mm. The final pressing force was 600 lb, and the final weights for the tablets were 50 mg, 45 mg, 42 mg, and 40 mg/tablet for Tablets A, B, C, and D, respectively.

Artificially stained bovine central incisors mounted in a resin were purchased from Dental Product Testing Therametric Technologies, Inc. Teeth having an L* value between 58 and 63 were selected for this study. Each of Tablets A, B, C, and D as prepared above was disintegrated separately in 20 mL of the mouthwash solution prepared as described above, such that the resulting four solutions contained NaDCC in an amount of 500 ppm, 250 ppm, 100 ppm, and 0 ppm, respectively. To disintegrate the tablets, the tablets and mouthwash were mixed and shaken for 90 seconds to ensure full disintegration of the tablet in the mouthwash. The liquid was then pooled into a tray and used to soak the stained teeth. Each tray was soaked for 1 minute, followed by about 20 seconds of rinsing with running tap water. After each treatment, L*, a*, and b* values were measured using a Spectroshade Micro instrument manufactured by Medical High Technology (MHT). The whitening performance is reported as the change in absolute W* value after treatments compared to baseline value. The higher the ΔW value, the whiter the tooth compared to the baseline measurement. The following formulae were used to calculate whiteness and the change in whiteness:

W*=(a*²+b*²+(L*−100)²)^{1/2}

ΔW=W*_{treated}−W*_{baseline}

Since the amount of sodium carbonate in each tablet was the same (40 mg), the final pH of the tablet/mouthwash solution of about 5.0 was similar for each of the four solutions. It was discovered, as shown below in Table 3, that mouthwash solutions plus NaDCC enhance whitening efficacy as compared to the mouthwash solution alone, which contained about 4% SAPP. Particularly, the whitening solution with 500 ppm NaDCC (the solution comprising Tablet A) gave a much faster whitening rate. For example, the combination of the mouthwash with 500 ppm NaDCC showed a high whitening efficacy (ΔW₅₀₀ₚₚₘ=−8.260) after 10 treatments. This whitening efficacy was larger than that from mouthwash alone with sodium bicarbonate treatment under the same conditions (ΔW_{MW}=−3.732) and compositions containing less NaDCC in the mouthwash solution (i.e., ΔW₂₅₀ₚₚₘ=−6.541; ΔW₁₀₀ₚₚₘ=−5.777). A similar whitening trend was found after 20 treatments. See Table 3 below. Since the tablet/mouthwash solutions were of similar pH, the enhanced whitening efficacy for formulae containing NaDCC is believed to be attributed to the formation of hypochlorite at the point of use.

TABLE 3
Whitening Efficacy (ΔW*) for In Vitro Treatments
on Stained Bovine Teeth

| | ΔW* after 10 | ΔW* after 20 |
|---|---|---|
| Solution | treatments | treatments |
| Mouthwash + | −8.260 ± 1.002 | −10.849 ± 1.137 |
| Tablet A (500 ppm | | |
| NaDCC) | | |
| Mouthwash + | −6.541 ± 1.386 | −8.046 ± 1.255 |
| Tablet B (250 ppm | | |
| NaDCC) | | |
| Mouthwash + | −5.777 ± 1.2 | −6.933 ± 1.778 |
| Tablet C (100 ppm | | |
| NaDCC) | | |
| Mouthwash + | −3.732 ± 1.404 | −4.714 ± 1.281 |
| Tablet D (0 | | |
| NaDCC) | | |

### Example 3—Antibacterial Study of NaDCC Solution

In order to screen for preliminary antibacterial efficacy, a NaDCC solution was subjected to an ATP-bacteria viability test. This method determines the number of viable bacterial cells in culture based on the quantification of adenosine triphosphate (ATP) present. ATP is a product of the catabolism of glucose (glycolysis) and aerobic respiration. This essential nucleoside triphosphate powers virtually all cell functions and is used by bacteria during cell division, the synthesis of nucleic acids and proteins, the transport of macromolecules, cell signaling, and bacterial motility. Given its importance, ATP signals the presence of metabolically active cells, and therefore it may be used to quantify cell viability. This method uses a luciferase reporter to measure the amount of cellular ATP present, hence the more effective antibacterial properties the composition has, the less ATP found.

In a reaction, luciferin is converted to oxiluciferin by the luciferase enzyme in the presence of ATP and oxygen. A direct relationship exists between the light output of the reaction and the number of metabolically active cells that makes this method ideal for the rapid screening of antimicrobial agents during the development of oral care formulas to establish their potential antibacterial efficacy. A salivary ATP assay was used to evaluate the antibacterial efficacy of NaDCC-containing tablets on bacterial viability of stimulated saliva samples.

In this assay, a mouthwash solution containing 0.075% cetylpyridinium chloride (CPC), a known antibacterial agent, was used as a positive control. An NaDCC tablet containing only NaDCC and weighing 20 mg was disintegrated in two phosphate buffered saline (PBS) solutions to yield both a 500 ppm NaDCC solution and a 1000 ppm NaDCC solution. Results showed that the NaDCC solutions (at both 500 ppm and 1000 ppm) exhibited statistically significant improvement in delivery of antibacterial properties compared to the 0.075% CPC solution, as shown below in Table 4, after a one-minute treatment. The treatment experiment was conducted two times for each of the two solutions and the control solution, and the results averaged. Lower viability translates into higher antibacterial properties. The data below in Table 4 was calculated using the Tukey Method with a 95% Confidence Interval for the mean.

TABLE 4
Intervals in Log10 of Viability v. Treatment

| Solution | Mean |
|---|---|
| 0.075% CPC solution | 5.061 ± 0.032 |
| 500 ppm NaDCC in | 4.139 ± 0.001 |
| PBS | |
| 1000 ppm NaDCC in | 3.530 ± 0.048 |
| PBS | |

### Example 4—pH-Dependent Bleaching Capability of NaDCC Solutions

Two different solutions, Solution A and Solution B, were prepared. Solution A contained 2% SAPP, 0.1% Lissamine green B dye, and 97.9% water. Solution B contained 2% tetrasodium pyrophosphate (TSPP), 0.1% Lissamine green B dye, and 97.9% water. Next, solutions with various pHs were obtained by mixing the two different solutions A and B, in different volume ratios (i.e., 1:0, 1:5, 1:1, 5:1, and 0:1). 20 mL of the resulting solutions were added to 10 mg NaDCC, and the time required to bleach away all of the Lissamine green B color was recorded.

As shown in Table 5 below, as the pH of the solution increased, the time required to bleach away Lissamine green B increased, indicating either the conversion of NaDCC to hypochlorite or oxidation of hypochlorite or both become more limited as the pH increases. In order to obtain whitening efficacy without damaging tooth enamel, NaDCC in oral care products may be used under weak acidic conditions.

TABLE 5
Bleaching Efficacy of NaDCC in Different pH Solutions

| Mix | | | Time to |
|---|---|---|---|
| Solution | V_{A}:V_{B} | pH | bleach |
| 1 | 1:0 | 4.6 | Instantaneous |
| 2 | 1:5 | 5.8 | 15 seconds |
| 3 | 1:1 | 7.4 | 8 minutes |
| 4 | 5:1 | 8.8 | 35 minutes |
| 5 | 0:1 | 10.5 | 80 minutes |

### Example 5—Disintegration Rates

Tablets containing 20% by weight NaDCC were disintegrated in mouthwash having a pH of 4.33 and disintegration rates were measured, as shown in Table 6 below. The tablets were prepared using a press and die set with a diameter of 5 mm. The final pressing force was 600 lbs, and the final weight for each tablet was 50 mg. The mouthwash formula is provided in Example 1, Table 1 above. Disintegration was based on the time required to fully disintegrate a tablet in 20 mL of the mouthwash.

TABLE 6
Disintegration rates of NaDCC tablets in mouthwash

| | | | | | | | | |
|---|---|---|---|---|---|---|---|---|
| Tablet E | Tablet F | Tablet G | Tablet H | Tablet I | Tablet J | Tablet K | Tablet L | Tablet M |

Ingredients (%)

| | | | | | | | | | |
|---|---|---|---|---|---|---|---|---|---|
| PVP-VA | 1 | 1 | 2.14 | 3 | 1 | 2.75 | 2.38 | 2.14 | 2.10 |
| Sodium | 67.4 | 73.04 | 71.98 | 74.6 | 73.88 | 67.25 | 66.62 | 71.98 | 75 |
| bicarbonate | | | | | | | | | |
| Magnesium | 1.5 | 1.22 | 1.08 | 1.21 | 0.65 | 1.5 | 0.5 | 1.08 | 1.5 |
| stearate | | | | | | | | | |
| Microcrystalline | 3 | 1 | 3 | 1.19 | 2.35 | 1 | 3 | 3 | 2.5 |
| cellulose | | | | | | | | | |
| Sodium acid | 7.1 | 3.74 | 1.81 | 0 | 2.11 | 7.5 | 7.5 | 1.81 | 0 |
| pyrophosphate | | | | | | | | | |

Disintegration time (seconds)

| | | | | | | | | | |
|---|---|---|---|---|---|---|---|---|---|
| Run #1 | 107 | 385 | 86 | 235 | 49 | 1196 | 120 | 86 | 210 |
| Run #2 | 115 | 402 | 83 | 228 | 41 | 1249 | 127 | 81 | 227 |
| Run #3 | 121 | 401 | 90 | 239 | 44 | 1268 | 118 | 88 | 199 |
| Average | 114 | 396 | 86 | 234 | 45 | 1238 | 122 | 85 | 212 |
| disintegration | | | | | | | | | |
| time (seconds) | | | | | | | | | |
| Ave. seconds | 465 | 183 | 493 | 345 | 534 | — | 457 | 494 | 367 |
| faster than | | | | | | | | | |
| Aquatabs ® | | | | | | | | | |

50 mg Aquatabs®, commercially available water-soluble disinfecting tablets containing NaDCC, were also disintegrated in the mouthwash formula provided above in Table 1. For Runs #1, #2, and #3, the disintegration time of Aquatabs® in 20 mL of mouthwash was 538 seconds, 607 second, and 591 seconds, respectively, for an average disintegration time of 579 seconds. Accordingly, all of Tablets E, F, G, H, I, K, L, and M had faster disintegration rates than Aquatabs® under similar disintegration conditions.

Next, tablets containing 20% by weight NaDCC were disintegrated in a mouthwash having a pH of 4.5, and the disintegration rates were measured, as shown in Table 7 below. As with the disintegration experiment discussed above, the tablets were prepared using a press and a dia set with a diameter of 5 mm. The final pressing force was 600 lbs, and the final weight for each tablet was 50 mg. Disintegration was based on the time required to fully disintegrate a tablet in 20 mL of the mouthwash.

TABLE 7
Disintegration rates of NaDCC tablets in mouthwash

| | | | | | | | |
|---|---|---|---|---|---|---|---|
| Tablet I | Tablet 2 | Tablet 3 | Tablet 4 | Tablet 5 | Tablet 6 | Tablet 7 | Tablet 8 |

Ingredients (%)

| | | | | | | | | |
|---|---|---|---|---|---|---|---|---|
| PVP-VA | 1 | 1 | 1 | 1 | 1 | 0 | 0 | 0 |
| PVP | 0 | 0 | 0 | 0 | 0 | 1 | 0 | 0 |
| Sodium | 73.88 | 76.24 | 76.69 | 76.79 | 76.89 | 76.89 | 77.89 | 80 |
| bicarbonate | | | | | | | | |
| Magnesium | 0.65 | 0.65 | 0.2 | 0.1 | 0 | 0 | 0 | 0 |
| stearate | | | | | | | | |
| Microcrystalline | 2.35 | 0 | 0 | 0 | 0 | 0 | 0 | 0 |
| cellulose | | | | | | | | |
| Sodium acid | 2.11 | 2.11 | 2.11 | 2.11 | 2.11 | 2.11 | 2.11 | 0 |
| pyrophosphate | | | | | | | | |

Disintegration time (seconds)

| | | | | | | | | |
|---|---|---|---|---|---|---|---|---|
| Run #1 | 60 | 211 | 120 | 30 | 10 | 8 | 11 | 120 |
| Run #2 | 61 | 142 | 180 | 27 | 7 | 9 | 9 | 150 |
| Run #3 | 60 | 107 | 150 | 27 | 10 | 10 | 10 | 180 |
| Average | 60 | 153 | 150 | 28 | 9 | 9 | 10 | 150 |
| disintegration | | | | | | | | |
| time (seconds) | | | | | | | | |
| Ave. seconds | 523 | 430 | 433 | 555 | 574 | 574 | 573 | 433 |
| faster than | | | | | | | | |
| Aquatabs ® | | | | | | | | |

50 mg Aquatabs® were also disintegrated in the same mouthwash having a pH of 4.5. For Runs #1, #2, and #3, the disintegration times of Aquatabs® in the mouthwash was 569 seconds, 600 seconds, and 580 seconds, respectively, for an average disintegration time of 583 seconds. Accordingly, all of Tablets I and 2-8 had faster disintegration rates than Aquatabs®.

The results indicate compositions that are free of magnesium stearate may have reduced disintegration times (i.e., increased disintegration rates). It is also shown that, by comparing Tablet I to Tablet 2, removing microcrystalline cellulose increased the disintegration rate, which is expected based on microcrystalline cellulose's known ability to assist in the disintegration of tablets. However, microcrystalline cellulose itself is not soluble and therefore may leave behind a residue in a mouthwash or other aqueous composition that consumers may find unacceptable. Accordingly, it may be advantageous to have a composition that, like Tablets 5-8, is free of microcrystalline cellulose. Finally, it is shown that water-soluble polymers such as PVP-VA and PVP and/or an effervescent acid such as sodium acid pyrophosphate may be removed from the composition and still result in a tablet having a reduced disintegration time, as shown by Tablets 7 and 8.

1. A solid composition for oral care comprising:
at least one whitening agent chosen from chloride isocyanurate and metal salts of hypochlorite;
at least one carbonate base present in the solid composition in an amount ranging from about 45% to about 95%, by weight relative to the total weight of the solid composition; and
at least one effervescent acid present in the solid composition in an amount ranging from about 0.01% to about 20%, by weight relative to the total weight of the solid composition.

2. The solid composition for oral care according to claim 1, wherein the at least one whitening agent is sodium dichloroisocyanurate.

3. The solid composition for oral care according to claim 1, wherein the at least one whitening agent is present in the solid composition in an amount ranging from about 0.5% to about 50%, by weight relative to the total weight of the solid composition.

4. The solid composition for oral care according to claim 1, wherein the at least one carbonate base is sodium bicarbonate.

5. The solid composition for oral care according to claim 1, wherein the at least one carbonate base is present in the solid composition in an amount ranging from about 65% to about 80%, by weight relative to the total weight of the solid composition.

6. The solid composition for oral care according to claim 1, wherein the at least one effervescent acid is sodium acid pyrophosphate.

7. The solid composition for oral care according to claim 1, wherein the solid composition is a tablet.

8. The solid composition for oral care according to claim 1, wherein the solid composition is free of microcrystalline cellulose.

9. The solid composition for oral care according to claim 1, wherein the solid composition is free of magnesium stearate.

10. The solid composition for oral care according to claim 1, wherein the solid composition is free of a water-soluble polymer.

11. The solid composition for oral care according to claim 1, wherein 50 mg of the solid composition is capable of disintegrating in 20 mL of an aqueous liquid composition in less than about 180 seconds.

12. The solid composition for oral care according to claim 1, wherein 50 mg of the solid composition is capable of disintegrating in 20 mL of an aqueous liquid composition in less than about 90 seconds.

13. The solid composition for oral care according to claim 1, wherein 50 mg of the solid composition is capable of disintegrating in 20 mL of an aqueous liquid composition in about 15 seconds or less.

14. A kit comprising:
a solid composition for oral care comprising at least one whitening agent chosen from chloride isocyanurate and metal salts of hypochlorite, at least one carbonate base present in the solid composition in an amount ranging from about 45% to about 95%, and at least one effervescent acid present in the solid composition in an amount ranging from about 0.01% to about 20%;
a container; and
an aqueous mouthwash composition inside the container.

15. The kit according to claim 14, further comprising a cup suitable for holding a specific amount of the aqueous mouthwash composition and a unit dose of the solid composition that is appropriate for the specific amount.

16. The kit according to claim 14, wherein the at least one whitening agent is sodium dichloroisocyanurate.

17. A method of whitening teeth, the method comprising:
mixing a solid composition for oral care comprising at least one whitening agent chosen from chloride isocyanurate and metal salts of hypochlorite, at least one carbonate base present in the solid composition in an amount ranging from about 45% to about 95%, and at least one effervescent acid present in the solid composition in an amount ranging from about 0.01% to about 20% with an aqueous liquid composition until the solid composition disintegrates to create a mouthwash comprising hypochlorite;
contacting the teeth with the mouthwash comprising hypochlorite; and
optionally expectorating the mouthwash comprising hypochlorite.

18. The method of claim 17, wherein 50 mg of the solid composition disintegrates in about 20 mL of the aqueous liquid composition in less than about 90 seconds.

19. The method of claim 17, wherein 50 mg of the solid composition disintegrates in about 20 mL of the aqueous liquid composition in about 15 seconds or less.

20. A solid composition for oral care comprising:
at least one whitening agent chosen from chloride isocyanurate and metal salts of hypochlorite; and
at least one carbonate base present in the solid composition in an amount ranging from about 45% to about 95%, by weight relative to the total weight of the solid composition,
wherein the solid composition is free of at least one effervescent acid.
